(19) United States
(12) Patent Application Publication
(10) Pub. No.: US 2007/0123783 A1
(43) Pub. Date: May 31, 2007

(54) Simplified physiological measurement device

(76) Inventor: Kuo-Yuan Chang, Shing-Tien City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/488,032

(22) Filed: Jul. 18, 2006

(30) Foreign Application Priority Data
Nov. 30, 2005 (TW) 094141988

(51) Int. Cl. A61B 5/00; A61B 5/02

(52) U.S. Cl. 600/485; 600/549; 600/500; 600/323; 600/300

(57) ABSTRACT

A simplified physiological measurement device utilizes a general port to receive power from an external electronic device and to transmit the signals obtained from physiological measurements to the external electronic device for calculation and display. Thereby, the mechanisms and electronic elements of the physiological measurement device can be obviously simplified, and the operational convenience can also be greatly promoted.

### BACKGROUND OF THE INVENTION

1. Field of the Invention

The present invention relates to a physiological measurement device, particularly to a physiological measurement device, which utilizes a general connecting port to obtain power, display, and calculation service provided by an external electronic device.

2. Description of the Related Art

Owing to the high material living standard of the modern society, the opportunity that modern people get a disease of civilization has greatly increased. According to some reports, the morbidity of hypertension has reached as high as one-fifth among adults over forty years old. Hypertension will harm brain, heart and kidney; for example, hypertension will incur brain vascular sclerosis, which is apt to bring about the hemorrhage or block of a blood vessel and result in apoplexy; hypertension may also incur ventricular hypertrophy, heart failure or angina pectoris, and a serious sufferer may die of myocardial infraction; and hypertension may also incur renal arterial sclerosis or uremia. Therefore, frequent blood pressure monitoring is very important and helpful to health.

Generally, the conventional blood pressure meters not only have the functions of measurement, calculation and display but also have batteries to provide power. If the conventional blood pressure meter can also be powered by alternating current simultaneously, it further needs a transformer. Therefore, the conventional blood pressure meters are often too bulky and heavy to carry about. Due to the built-in calculation and display functions, the fabrication cost of the conventional blood pressure meters is also high.

With the advance of science and technology, many function-integration computers, PDA's (Personal Digital Assistant) and potable communication devices (such as mobile phones) are emerging. As the abovementioned electronic devices have to implement their functions, all of them should have calculation, display functions and power-supply elements. In comparison with the abovementioned electronic devices, it is found that there is some functional commonness between a general blood pressure meter and the abovementioned electronic devices.

Under the tendency of fabricating slim and lightweight portable electronic products, how to decrease the elements of a physiological measurement device but maintain the basic functions thereof has become an important subject. Besides, the convenience of operation and the future usableness of measurement data are also important factors in designing a physiological measurement device.

### SUMMARY OF THE INVENTION

One of objects of the present invention is to provide a physiological measurement device to simply the design of physiological measurement devices, wherein calculation, display and power-supply units are transferred to another electronic device, such as a personal computer, a PDA (Personal Digital Assistant) and a portable communication device, which can be electrically coupled to the physiological measurement device, so that the quantity of the elements and the complexity of the circuits may be reduced.

Another object of the present invention is to provide a physiological measurement device to increase the usage flexibility of the measurement data of physiological measurement devices, wherein the physiological measurement device of the present invention has a USB (Universal Serial Bus) port connecting with a USB cable to transmit the measurement data to an external electronic device in real time for subsequent processing, calculating and displaying.

To achieve the abovementioned objects, an embodiment of the present invention proposes a physiological measurement device, which utilizes a signal cable to connect with an external electronic device and comprises: a signal detection unit, contacting a living body, and detecting an analog physiological signal of the living body; a signal conversion unit, coupled to the signal detection unit, receiving the analog physiological signal, and transforming the analog physiological signal into a digital physiological signal; and a Connecting port, coupled to the signal detection unit and the signal conversion unit, receiving power from an external electronic device via the signal cable to provide power for the signal detection unit and the signal conversion unit, and outputting the digital physiological signal to the external electronic device via the signal cable.

### BRIEF DESCRIPTION OF THE DRAWINGS

FIG. 1 is a block diagram schematically showing the system of the physiological measurement device according to a first embodiment of the present invention.

FIG. 2 is a block diagram schematically showing the system of the physiological measurement device according to a second embodiment of the present invention.

### DETAILED DESCRIPTION OF THE INVENTION

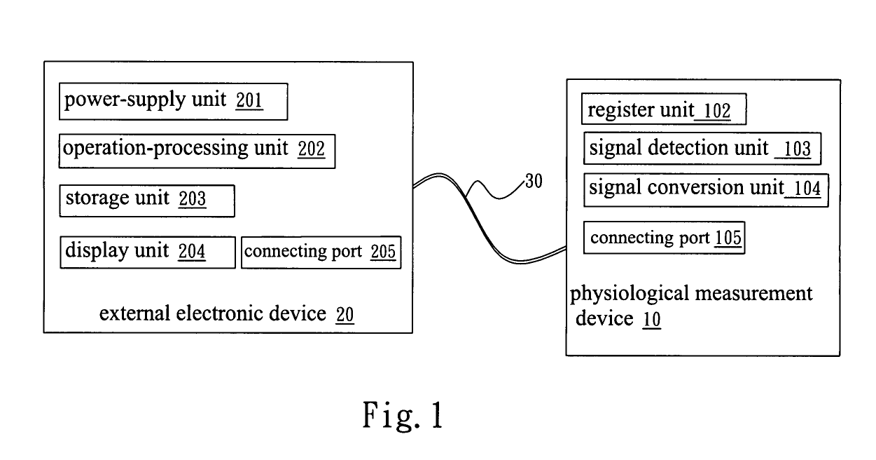

Refer to FIG. 1 a block diagram schematically showing the system of the physiological measurement device according to a first embodiment of the present invention. According to the embodiment of the present invention, the physiological measurement device 10 includes: a register unit 102, a signal detection unit 103, a signal conversion unit 104, and a connecting port 105. The signal detection unit 103 contacts a living body and detects an analog physiological signal of the living body, such as blood pressure (including hypertension and hypotension), cardiac pulsation, oxygen in blood, and body temperature. The signal conversion unit 104 is coupled to the signal detection unit 103 to receive the analog physiological signal detected by the signal detection unit 103 and transform the analog physiological signal into a digital physiological signal. In one embodiment, the signal conversion unit 104 includes an A/D (analog/digital) converter. The connecting port 105 is coupled to the signal detection unit 103, the signal conversion unit 104 and a signal cable 30 to receive power via the signal cable 30 to provide power for the signal detection unit 103 and the signal conversion unit 104 and output the digital physiological signal of the signal conversion unit 104 to an external electronic device 20 via the signal cable 30. It is noted that the embodiments of the present invention are not limited to the connecting port 105. Any connecting port in a general communication protocol may be configured to a port in the present invention. Furthermore, the connecting port applied to the present invention may have a specific controller on one terminal.

The spirit of the present invention is to utilize the existing components and functions of general external electronic devices 20, such as a power supply and a processor, to support the physiological measurement device 10 and reduce the volume and cost of the physiological measurement device 10. The physiological measurement device 10 may be a blood pressure meter, an oximeter, a clinical thermometer, a blood glucose meter, or a combination of them. The physiological measurement device 10 may be fixed to the arm, the wrist, or the finger for measuring. The external electronic device 20 may be a personal computer, a PDA, or a portable communication device and generally has a power-supply unit 201, an operation-processing unit 202, a storage unit 203, a display unit 204, and a connecting port 205. The connecting port 205 may be connected to the connecting port 105 of the physiological measurement device 10 via the signal cable 30, and the power-supply unit 201 of the external electronic device 20 can supply power, such a 5 V DC power, to the physiological measurement device 10 via the signal cable 30. The operation-processing unit 202 of the external electronic device 20 may also utilize the signal cable 30 to send instructions to the signal detection unit 103 of the physiological measurement device 10 and instruct the signal detection unit 103 to execute a measurement process. The operation-processing unit 202 of the external electronic device 20, such as a CPU (Central Processing Unit), may also utilize the signal cable 30 to receive the digital physiological signal of the physiological measurement device 10; the operation-processing unit 202 processes the received digital physiological signal and then presents the calculation results on the display unit 204 and stores the calculation results in the storage unit 203 (such as a memory).

Below, a blood pressure measurement is used to exemplify the present invention. The signal detection unit 103 of the physiological measurement device 10 may further include: a pump module and a sensor module (not shown in the drawings); two connecting ports 105 and 205 are interconnected via the signal cable 30, and the external electronic device 20 instructs the pump module to pump air until a preset pressure is reached, and then the air is gradually released, and the sensor module begins to detect a series of pulsations of the blood pressure. What the physiological measurement device 10 of the present invention needs to do are only collecting the analog physiological signal and transforming the analog physiological signal into a digital physiological signal. Then, the digital physiological signal is transferred to the external electronic device 20 via the signal cable 30. Therefrom, the external electronic device 20 takes over the succeeding tasks, and the digital physiological signal will be processed, analyzed and displayed by the external electronic device 20. According to those discussed above, the external electronic device 20 has to possess some basic components to implement the required functions; contrarily, the physiological measurement device 10 is intended to be simple, slim, lightweight, and low-cost as much as possible. Therefore, the present invention utilizes the connecting ports, such as USB port, and the signal cable, such as a USB signal cable, to integrate the physiological measurement device 10 and the external electronic device 20 and utilizes the existing components of the external electronic device 20 to provide the power, calculation and display services required by the physiological measurement device 10, so that the volume and fabrication cost of the physiological measurement device 10 can be obviously reduced. Besides, the detected physiological data can be stored in the external electronic device 20 for future use.

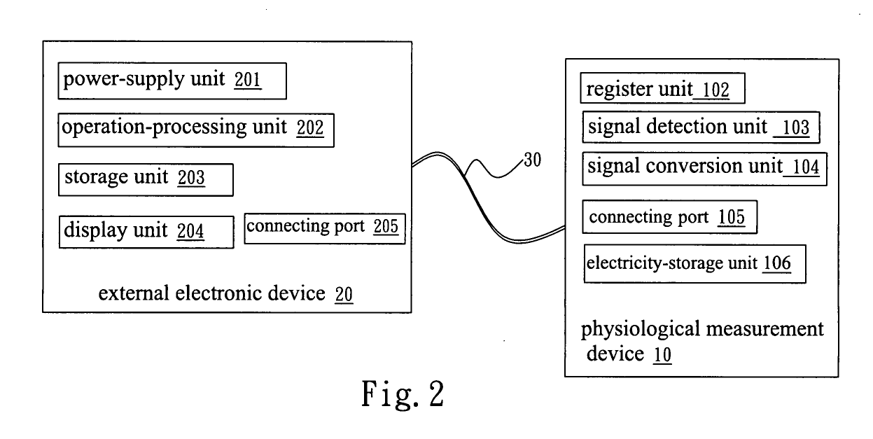

Refer to FIG. 2 a block diagram schematically showing the system of the physiological measurement device according to a second embodiment of the present invention. In comparison with the first embodiment shown in FIG. 1, the physiological measurement device 10 of the second embodiment further comprises an electricity-storage unit 106, such as a storage capacitor. When the physiological measurement device 10 receives power from the external electronic device 20 via the signal cable 30 and the connecting port 105, the electricity-storage unit 106 can also be charged. Once the external electronic device 20 suddenly stops power supply because of some irresistible factor, the physiological measurement device 10 can still execute measurements via the power of the electricity-storage unit 106.

According to those described above, the physiological measurement device, which embodies the spirit of the present invention, may be a blood pressure meter, a clinical thermometer, or a blood glucose meter, and the physiological measurement device of the present invention has a general connecting port functioning as an I/O port, which receives required power and outputs detected physiological signals. The power-supply, processing/calculating, and display components, which the conventional physiological measurement device must possess, may be omitted in the physiological measurement device of the present invention. Thus, the physiological measurement device of the present invention is slim, lightweight and low-cost. Further, in the present invention, the detected physiological signal can be transmitted to an external electronic device in real time; and the user can utilize the external electronic device to process the physiological data and then display the processed physiological data. The user may also utilize the external electronic device to store the physiological data so that he can flexibly use the physiological data later.

What is claimed is:

1. A physiological measurement device, utilizing a signal cable to connect with an external electronic device, the physic measurement device comprising:
a signal detection unit contacting a living body and detecting an analog physiological signal of said living body;
a signal conversion unit coupled to said signal detection unit, receiving said analog physiological signal, and transforming said analog physiological signal into a digital physiological signal; and
a connecting port coupled to said signal detection unit and said signal conversion unit, receiving power from said external electronic device via said signal cable to provide power for said signal detection unit and said signal conversion unit, and outputting said digital physiological signal to said external electronic device via said signal cable.

2. The physiological measurement device according to claim 1, comprising a blood pressure meter.

3. The physiological measurement device according to claim 2, wherein said signal detection unit further comprises:
a sensor module, sensing said analog physiological signal of said living body; and
a pump module, executing air-pumping and air-releasing actions when said pump module receives a corresponding instruction via said Connecting port and said signal cable.

4. The physiological measurement device according to claim 1, comprising used to measure heart beating.

5. The physiological measurement device according to claim 1, which is a clinical thermometer.

6. The physiological measurement device according to claim 1, comprising an oximeter.

7. The physiological measurement device according to claim 1, comprising a blood glucose meter.

8. The physiological measurement device according to claim 1, comprising used to measure cardiac pulsation.

9. The physiological measurement device according to claim 1, wherein said signal detection unit executes a measurement step when said signal detection unit receives a corresponding instruction via said connecting port and said signal cable.

10. The physiological measurement device according to claim 1, wherein said connecting port is a universal serial bus port.

11. The physiological measurement device according to claim 1, further comprising a register unit coupled to said connecting port and said signal detection unit and used to temporarily store an electrical signal from said signal cable.

12. The physiological measurement device according to claim 1, further comprising an electricity-storage unit coupled to said connecting port, said signal detection unit, and said signal conversion unit and used to store the electricity coming from said external electronic device and transmitted via said signal cable.

13. The physiological measurement device according to claim 1, wherein said signal conversion unit is an analog/digital converter.

14. An electronic blood pressure meter, comprising:
a pump module executing air-pumping and air-releasing actions to apply different pressures to a contacted living body when said pump module receives a corresponding instruction from an external electronic device;
a sensor detecting a physiological signal of said contacted living body when said pump module applies different pressures to said contacted living body;
an analog/digital converter coupled to said sensor and transforming said physiological signal into a digital physiological signal; and
an universal serial bus (USB) port coupled to said pump module and said analog/digital converter, receiving power and instructions from said external electronic device, and providing said power and said instructions for said pump module and said analog/digital converter.

15. The electronic blood pressure meter according to claim 14, wherein said USB port also outputs said digital physiological signal to said external electronic device.

16. The electronic blood pressure meter according to claim 14, further comprising a storage capacitor coupled to said USB port and storing the electricity received from said external electronic device.

17. The electronic blood pressure meter according to claim 14, comprising fixed to the arm of said contacted living body.

18. The electronic blood pressure meter according to claim 14, comprising fixed to a wrist of said contacted living body.

19. The electronic blood pressure meter according to claim 14, comprising fixed to a finger of said contacted living body.
